(19) United States
(12) Patent Application Publication
(10) Pub. No.: US 2005/0010105 A1
(43) Pub. Date: Jan. 13, 2005

(54) Method and system for Coronary arterial intervention

(76) Inventor: Jasbir S. Sra, Pewaukee, WI (US)

Correspondence Address: JANSSON, SHUPE & MUNGER, LTD, 245 MAIN STREET, RACINE, WI, 53403, US

(21) Appl. No.: 10/882,517

(22) Filed: Jul. 1, 2004

Related U.S. Application Data
(60) Provisional application No. 60/484,045, filed on Jul. 1, 2003.

(51) Int. Cl. A61B 5/05

(52) U.S. Cl. 600/424

(57) ABSTRACT

A method is provided for arterial intervention on a patient that has the steps of obtaining digital image data of the patient's artery from a medical imaging system where the artery has lesions arising from arterial disease, generating a 3D model from this image data, registering the 3D model to an image of the artery that has been visualized in real-time upon an interventional system, navigating an angioplasty delivery system to the artery utilizing this registered 3D model, and using the angioplasty delivery system to treat the artery. Preferably, the digital image data is cardiac image data, the artery is a coronary artery, and the angioplasty delivery system is a stent and stent delivery system. In another aspect of this invention, it provides a system for arterial intervention that has a medical imaging system for obtaining digital image data of at least one of the patient's arteries, an image generation system for generating a 3D model from the image data, an interventional system for visualizing an image of the artery in real-time, a workstation for registering the 3D model to this image, and an angioplasty delivery system that can be navigated to the artery utilizing the registered 3D model.

### RELATED APPLICATION

This application claims the benefit of U.S. Provisional Application No. 60/484,045,

### FIELD OF THE INVENTION

This invention relates generally to methods and systems for cardiac interventional treatment and, in particular, to methods and systems for coronary artery intervention and for the planning of such intervention.

### BACKGROUND OF THE INVENTION

Over 12 million people in the United States alone have coronary artery disease (CAD). Over 1 million new and recurrent cases of coronary attacks (i.e., angina, heart attacks) are diagnosed per year. Over 500,000 deaths per year are related to CAD.

Angioplasty is an effective way of opening up blocked coronary arteries. Over 50 percent of all angioplasties are performed to clear out coronary arteries and the remainder of the procedures are for other arteries, such as those in the legs. Initially, angioplasty only involved using a balloon catheter to open the blocked artery, but most of the angioplasties done today also include placement of small metallic devices called “stents”. It is estimated that over 1 million stents were placed in the year 2002. The stent is usually collapsed to a small diameter and put over a balloon catheter. It is then moved into the area of blockage at which location the stent is expanded to form a scaffold. The stent remains in the artery permanently. Stent placement may be used in conjunction with or in place of an angioplasty. Presently, the use of a stent depends on the presence of certain features in the blocked artery. Stenting now represents 70-90 percent of the procedures done to open coronary arteries. Reclosure or restenosis is a problem with the stent procedure. New types of stents which are covered with drugs can reduce the incidence of restenosis somewhat.

The prevention of restenosis post stent placement, however, starts at the point of stent implantation. An understanding of the science of appropriate stent placement is thus crucial. A method and system for coronary artery intervention planning in which 3D imaging can be used to identify precisely the extent and degree of CAD as well as registration of these images and navigation of delivery tools to the site of stent placement should help eliminate the flaws in the current system and improve the efficacy and safety of stent placement or angioplasty.

It is an object of this invention to provide an improved method and system for arterial intervention treatment that overcomes some of the problems and shortcomings in the prior art, including those referred to above.

### SUMMARY OF THE INVENTION

One aspect of this invention provides a method for arterial intervention on a patient that include the steps of obtaining digital image data of the patient's artery from a medical imaging system where the artery has lesions arising from arterial disease, generating a 3D model from this image data, registering the 3D model to an image of the artery that has been visualized in real-time upon an interventional system, navigating an angioplasty delivery system to the artery utilizing this registered 3D model, and using the angioplasty delivery system to treat the artery.

In a desirable embodiment, the medical imaging system is a computer tomography (CT) system. Also preferred is where the method includes the step of visualizing the 3D model over a computer workstation of the interventional system.

One very preferred embodiment finds the digital image data to be cardiac image data and the artery to be a coronary artery. More desirable is when the step of generating the 3D model from the image data uses a protocol optimized for 3D imaging of the coronary artery. Most preferred is where the angioplasty delivery system is a stent and stent delivery system. Highly preferred is where the stent and stent delivery system are then visualized in real-time over a computer workstation on the interventional system.

Certain exemplary embodiments are where the interventional system is a fluoroscopic system. Also highly desired are embodiments where the method includes as well the step of visualizing the 3D model on a computer workstation so that the size, orientation and contour of the coronary artery can be assessed. Most preferred is where endocardial views of the coronary artery are generated from the cardiac image data so that these views can be seen simultaneously with the 3D model.

Another desired embodiment is where the image data obtained also includes at least one ventricle of the heart so that when the 3D model is visualized on a computer workstation of the interventional system, the structure and function of the ventricle can be assessed.

In another aspect of this invention, a system is provided for arterial intervention on a patient that has a medical imaging system for obtaining digital image data of at least one of the patient's arteries, an image generation system for generating a 3D model from the image data, an interventional system for visualizing an image of the artery in real-time, a workstation for registering the 3D model to this image, and an angioplasty delivery system that can be navigated to the artery utilizing the registered 3D model.

A desirable embodiment is where the medical imaging system is a computer tomography (CT) system. Also preferred is where the digital image data is cardiac image data and the artery is a coronary artery. Most preferred is when the angioplasty delivery system is a stent and stent delivery system.

Another aspect of this invention finds a method for planning arterial intervention having the steps of obtaining digital image data of an artery of a patient from a medical imaging system, generating a 3D model from this image data, registering the 3D model to an image of the artery that is visualized in real-time upon an interventional system, and visualizing this registered 3D model on the interventional system.

In another aspect of the invention, a system is provided for planning arterial intervention on a patient. This system includes a medical imaging system for obtaining digital cardiac image data of the patient's artery, an image generation system for generating a 3D model from this data, an interventional system for visualizing an image of the artery in real-time, and a workstation for registering the 3D model to this image and for also then visualizing the registered 3D model upon the interventional system.

### BRIEF DESCRIPTION OF THE DRAWINGS

FIG. 1 is a schematic outline of planning coronary artery intervention.

FIG. 2 is a flow diagram of a method for coronary artery intervention.

### DETAILED DESCRIPTION OF PREFERRED EMBODIMENTS

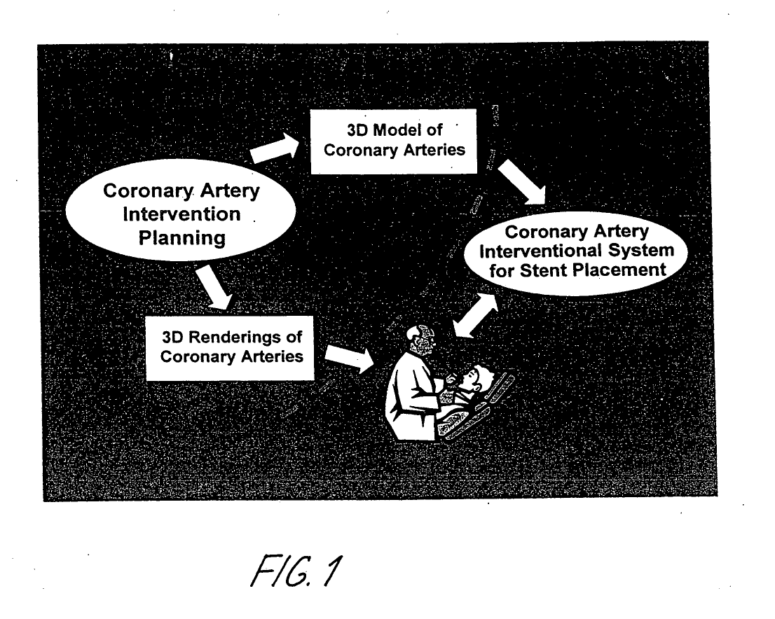

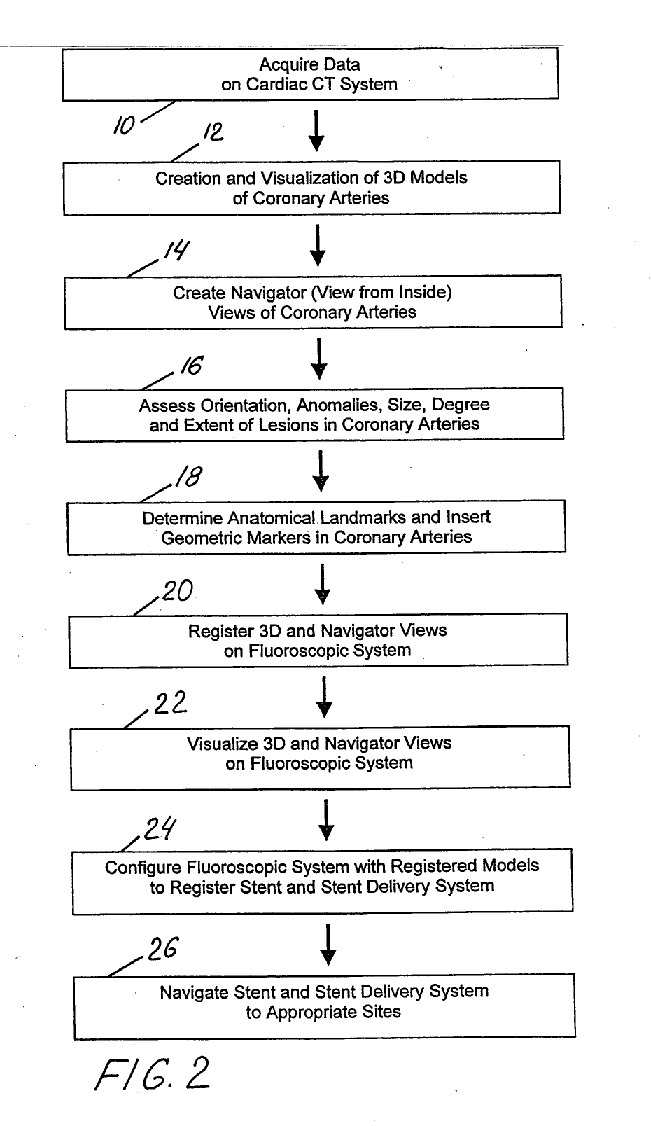

FIGS. 1-2 illustrate a method and system for planning coronary artery intervention in a patient with coronary artery disease. The embodiment shown enables a cardiologist to plan in advance a desired approach for stent placement. Using CT imaging, detailed 3D and endocardial views (i.e., navigator or views from the inside) of the coronary arteries are obtained. The cardiologist can identify the orientation, size, anomalies and extent of blockage in the coronary arteries to be targeted. Furthermore, registration of appropriate images and real-time navigation of a stent delivery system and stent enables exact sites to be targeted, making the procedure simpler and more efficacious while decreasing the risk of complications.

Although the embodiments illustrated hereinafter are described in the context of a CT imaging system, it will be appreciated that other imaging systems known in the art, such as MRI and ultrasound, are also contemplated with regards to planning for coronary artery intervention. Similarly, although the interventional system is described in the context of fluoroscopy and a computer workstation, other interventional system are also contemplated. In addition to coronary artery anatomy, the function of the ventricles could also be imaged, registered and visualized. Furthermore, it will be appreciated that, although the exemplary embodiments illustrated hereinafter are described in the context of stent placement in the coronary arteries, other systems such as angioplasty balloon and other arteries, such as those in the legs, kidneys, carotids or other organs, are also contemplated.

There is shown in FIG. 1 a schematic overview of an exemplary method for coronary artery intervention planning and system for stent placement. Imaging is preferably obtained using a CT system. The acquired data is registered with fluoroscopic system, which is also configured to register and visualize real-time navigation of the stent delivery system and the stent.

Referring now to FIG. 2, there is shown a detailed overview of the method for coronary artery intervention. As shown in step 10, the CT system is used to acquire data of the coronary arteries. The CT imaging system is automated to acquire data of the coronary arteries and other structures such as the ventricles. A continuous sequence of consecutive images is collected from a volume of patient's data. A shorter scanning time using a faster scanner and synchronization of the CT scanning with the QRS on the ECG signal will reduce the motion artifacts in a beating organ like the heart. The ability to collect a volume of data in a short acquisition time allows reconstruction of images which are true geometric depictions making them easier to understand.

In step 12, the dataset acquired by the CT image system is segmented and a 3D model of the coronaries is generated using protocols optimized for the coronary arteries. The 3D models of the coronary arteries are then visualized.

As shown in step 14, the coronary arteries are visualized using 3D surface and/or volume rendering to create 3D models of the coronary arteries. A post-processing software is used to create navigator (view from inside) views of the coronary arteries.

In the method of interventional planning for coronary artery disease, once the 3D images and navigator views are visualized as shown in step 10, orientation, size, contour and any anomalies of the coronary arteries are identified as indicated at step 16. The extent and degree of the lesions are also identified.

Subsequently, as illustrated in step 18, one or more anatomical landmarks are identified over the coronary arteries. Explicit geometric markers are then inserted into the volume at landmarks of interest, at which time the markers may be visualized in a translucent fashion. The specific images (Dicom images, video clips, films, multimedia) are saved as desired for subsequent reference during the coronary artery intervention planning. The apparatus for database storage may include hard drives, floppy diskettes, CD Roms or other storage mediums. A predetermined computer program will allow execution of storage and subsequent exportation of these images.

As shown in step 20, the saved views are then exported and registered with the fluoroscopic system. The transfer of 3D model and navigator views can occur in several formats such as Dicom format or object. Other formats such as geometric wire mesh model or additional formats known in the art can be used for exportation of images to the fluoroscopic system for the registration process. The exportation of images in real-time or from a stored database will occur using predetermined execution formats over the transmission media. A CT scan can depict detailed images of the coronary arteries. Integration of these images with a fluoroscopic system can significantly improve the efficacy and safety of planning for coronary artery intervention using a stent placement.

The registration method transforms the coordinates in the CT image into the coordinates in the fluoroscopic system. The degree of interaction between the two images may be interactive, semi-automatic and/or automatic. The interactive method needs human interference for determination of transformation. In the semi-automatic method, a computer determines the transformation, while user interaction is required for the selection of image properties to be used in the registration, starting or stopping of the matching procedure. Automatic methods need no human interaction.

Information from the CT will thus be integrated with the fluoroscopic system. One or more predetermined anatomical landmarks will be used for registration with the interventional system. These points can be seen separately from the rest of the coronary arteries. During the interventional procedure a similar point or points are taken and coordinated with the points taken on the CT images. Once these coordinates are locked in between the CT image and the fluoroscopic system or other interventional system, the 3D image and navigator views can be seen in different views on the interventional system as indicated at step 22. Multiple views can be seen sequentially or simultaneously.

As shown in step 24 of FIG. 2, the invention further involves the location of the stent and the stent delivery system over the fluoroscopic system and/or other intervention system. The fluoroscopic system is configured to locate the stent and the stent delivery system to be localized over the system. The stent delivery system and stent are then navigated to the appropriate site as illustrated at step 26.

A more detailed 3D geometric representation of the coronary arteries increases the precision of coronary stent placement by providing contour, position, orientation and dimensions (e.g., circumference), degree and extent of lesions of the coronary arteries.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A method for arterial intervention on a patient comprising:
obtaining digital image data of at least one artery of the patient from a medical imaging system, the artery having lesions arising from arterial disease;
generating a 3D model from the image data;
registering the 3D model to an image of the artery that is visualized in real-time upon an interventional system;
navigating an angioplasty delivery system to the artery utilizing the registered 3D model; and
using the angioplasty delivery system to treat the artery.

2. The method of claim 1 wherein the medical imaging system is a computer tomography (CT) system.

3. The method of claim 1 further comprising the step of visualizing the 3D model over a computer workstation of the interventional system.

4. The method of claim 1 wherein the digital image data is cardiac image data and the artery is a coronary artery.

5. The method of claim 4 wherein the angioplasty delivery system is a stent and stent delivery system.

6. The method of claim 5 wherein generating a 3D model from the image data comprises using a protocol optimized for 3D imaging of the coronary artery.

7. The method of claim 5 wherein the interventional system is a fluoroscopic system.

8. The method of claim 5 further comprising the step of visualizing the 3D model on a computer workstation of the interventional system whereby the size, orientation and contour of the coronary artery can be assessed.

9. The method of claim 8 further comprising the steps of generating endocardial views of the coronary artery from the cardiac image data and visualizing the endocardial views simultaneously with the 3D model, whereby the degree and extent of the lesions can be identified.

10. The method of claim 5 wherein the cardiac image data obtained includes at least one ventricle and the 3D model is visualized on a computer workstation of the interventional system whereby the structure and function of the ventricle can be assessed.

11. The method of claim 5 further comprising the step of visualizing the stent and stent delivery system in real-time over a computer workstation of the interventional system.

12. A system for arterial intervention on a patient comprising:
a medical imaging system for obtaining digital image data of at least one artery of the patient, the artery having lesions arising from arterial disease;
an image generation system for generating a 3D model from the image data;
an interventional system for visualizing an image of the artery in real-time;
a workstation for registering the 3D model to the image; and
an angioplasty delivery system, wherein the angioplasty delivery system is navigated to the artery utilizing the registered 3D model.

13. The system of claim 12 wherein the medical imaging system is a computer tomography (CT) system.

14. The system of claim 12 wherein the digital image data is cardiac image data and the artery is a coronary artery.

15. The system of claim 14 wherein the angioplasty delivery system is a stent and stent delivery system.

16. The system of claim 15 wherein the interventional system is a fluoroscopic system.

17. The system of claim 15 wherein the workstation also visualizes the registered 3D model and the stent and stent delivery system upon the interventional system, whereby the stent and stent delivery system is viewed in real-time over the 3D model.

18. The system of claim 17 wherein the 3D model further includes endocardial views of the coronary artery and the endocardial views are visualized simultaneously with the 3D model upon the interventional system.

19. A method for planning arterial intervention on a patient comprising:
obtaining digital image data of at least one artery of the patient from a medical imaging system, the artery having lesions arising from arterial disease;
generating a 3D model from the image data;
registering the 3D model to an image of the artery visualized in real-time upon an interventional system; and
visualizing the registered 3D model on the interventional system.

20. A system for planning arterial intervention on a patient comprising:
a medical imaging system for obtaining digital cardiac image data of at least one artery of the patient, the artery having lesions arising from arterial disease;
an image generation system for generating a 3D model from the image data;
an interventional system for visualizing an image of the artery in real-time; and
a workstation for registering the 3D model to the image and for visualizing the registered 3D model upon the interventional system.
